(19) United States
(12) Patent Application Publication
(10) Pub. No.: US 2005/0186280 A1
(43) Pub. Date: Aug. 25, 2005

(54) Method for stabilizing a composition having a biologically active epidermal growth factor as an active ingredient

(75) Inventors: Jeong-Hwa Yang, Kunpo (KR); Jang-Won Lee, Seoul (KR); Mi-Young Shon, Yongin-city (KR); Young-Jun Kim, Seoul (KR)

Correspondence Address: BIRCH STEWART KOLASCH & BIRCH, PO BOX 747, FALLS CHURCH, VA, 22040-0747, US

(73) Assignee: Daewoong Pharm. Co., Ltd.

(21) Appl. No.: 11/113,075

(22) Filed: Apr. 25, 2005

Related U.S. Application Data
(63) Continuation of application No. 09/958,116, filed on Oct. 4, 2001.

(30) Foreign Application Priority Data
Feb. 21, 2000 (KR) 2000-8116

(51) Int. Cl. A61K 38/18; A61K 9/14

(52) U.S. Cl. 424/486; 514/12

(57) ABSTRACT

A method for stabilizing a composition having a biologically active EGF as an active ingredient includes the step of adding a carboxyvinyl polymer as a stabilizer of the EGF. The content of the EGF is within the range of 0.1 to 1000 μg/g on the basis of the total weight of the composition, and the content of the carboxyvinyl polymer is within the range of 0.001 to 50 (w/w) % on the basis of the total weight of the composition.

### TECHNICAL FIELD

The present invention relates to a stable composition comprising epidermal growth factor (hereinafter referred to as “EGF”) as an active ingredient. More specifically, the present invention relates to a stable composition which comprises EGF having a biological activity and a carboxyvinyl polymer capable of being significantly increased stability of EGF in an aqueous solution as a base.

EGF(known as urogastrone) is a polypeptide having a molecular weight of 6045 which consists of 53 amino acid residues and includes three of disulfide bonds. EGF is known as a wound healing agent for the skin and is cornea and a gastric ulcer healing agent because it represents a good activity for stimulating mitosis of various cells including epidermal and messenchymal cells and growth thereof and controlling secretion of gastric acid. (U.S. Pat. No. 140,998; Carpenter, Experimental Cell Research, 164:1-10, 1986).

Although EGF shows a good activity for simulating differentiation of epidermal cells in vitro, it is very difficult that topical formulation containing EGF is developed to treat wounds of the skin and cornea for the reason that EGF has only a little effect in treating wounds when it is clinically applied to wounds.

EGF is biologically unstable and physicochemically non-homogenous so that its healing effects are not sufficient and its decomposition products may induce allergic reactions. Accordingly EGF cannot exhibit sufficient effects for treating wounds in an application to a living body. EGF is very unstable at the room temperature, particularly in the presence of moisture. Although a lag time is required about 8 to 12 hours for DNA synthesis on wounds, EGF has a very short half-life of about 1 hour not to get the desired effects. Furthermore, EGF is physicochemically denatured at the room temperature and even in the state of cold storage when it is stored for a long time. When EGF is applied on the skin, EGF loses biological activity resulting from denaturation, decomposition, condensation and precipitation of EGF due to proteolytic enzymes to exist in wounds (Manning et al., Pharmaceutical Res ., 6:903-917, 1989).

In order to overcome biological unstableness of EGF and provide its desired wound healing effect, it is reported that EGF is continuously applied on wounds during initial few days of treatment which are most important time for wounds healing so as to constantly maintain an effective level of EGF (Frankline et al., J. Lab. Clin. Med ., 108:103-108, 1986). In this regard, some sustained EGF-releasing formulations have been studied, which can continuously provide EGF to wounds.

As a result, U.S. Pat. No. 4,944,948 discloses the EGF/liposome gel formulations which continuously provide EGF to wounds using neutral phospholipids, negative-charged phospholipids and cholesterol; and EP Publication No. 312208 discloses the aqueous formulation being able to continuously release EGF which comprises pharmaceutically acceptable various water-soluble or water-swellable polymer as a base. However, although the above-mentioned prior arts disclose the formulations which can continuously release EGF for 12 hours or more, they are unsuitable for producing in industrial fields because these formulations are unstable in long-term storage. Therefore, it has been required that a biological activity of EGF is maintained for a long time and a physicochemical stability thereof such as purity and homogeneity as well in order to provide EGF sufficient wounds healing effect as a medicine.

As a method to maintain physicochemical stability of EGF and inhibit a decrease of EGF activity, EP Publication No. 205051 provides the pharmaceutical composition in the form of a cream for dermal and ophthalmic use, which comprises 0.0001-0.005% (w/w) of EGF, 1-10% (w/w) of surfactants, 5-45% (w/w) of fatty substances and 0.3-0.8% (w/w) of preservatives. EP Publication No. 267015 and U.S. Pat. No. 4,717,717 provides the compositions containing EGF stabilized by an addition of a water-soluble cellulose derivative to EGF. Also EP Publication No. 398615 and U.S. Pat. No. 5,130,298 provide the methods for stabilizing EGF by mixing EGF with a pharmaceutically acceptable metal cation such as zinc which is capable of preventing the degradation of EGF in aqueous solution since EGF is ionically bound with zinc.

However, although the above-mentioned stabilizers are added, the stability of EGF is maintained for about two months at 4° C. Therefore, when the topical formulation of EGF for the skin is clinically applied to wounds, they are unsuitable for utilizing in industrial fields since they have a little healing effect for wounds and the reduced stability of the formulation.

Accordingly, it is very desirable to develop the formulated preparation of EGF useful for treating incurable pathology and so on such as dermal ulcer or corneal injure in the state of no special treating agent, which sufficiently exhibit the wound-healing effects, has a protected EGF against a loss of biological activity and quickly delivers EGF from the carrier to wounds when it is applied.

Thus, the present inventors have conducted numerous studies to develop the topical preparation of EGF which has a sufficient wound-healing effect and a good stability. As a result, we have found that the topical preparation comprising EGF as an active ingredient and acidic polymer such as carboxyvinyl polymer as a base can exhibit the desired good wound-healing effect and significant stability as compared with the prior arts using a base such as cellulose based polymer or neutral polymer.

### DISCLOSURE OF THE INVENTION

It is therefore an object of the present invention to provide a biologically and physicochemically stable composition containing EGF, which comprises EGF as an active ingredient and a carboxyvinyl polymer as a base.

The composition according to the present invention comprises EGF as an active ingredient and a carboxyvinyl polymer as a base. EGF as an active ingredient may be isolated from natural sources or produced using recombinant DNA techniques. The content of EGF in the composition is within the range of 0.001 to 1,000 μg/g on the basis of the total weight of the preparation, preferably in the range of 0.1 to 100 μg/g such that EGF is pharmacologically effective. The pH of the composition according to the present invention is preferably in the range of 4 to 8, more preferably in the range of 5 to 7 in order to keep EGF dissolved without denaturation.

A carboxyvinyl polymer which is used as a base in the present invention is a homopolymer having molecular weight of 1×10⁶ to 4×10⁶. The carboxyvinyl polymer, which is a cross-linked product of acrylic acid and aryl sucrose, is an acidic polymer indicating pH of 2.5 to 3.0 when it is dispersed in 1% aqueous solution. It has the wide range of viscosity even in a low concentration of less than 1% so that it is widely used as a base to suspension for oral, lotion, cream and gel preparation Furthermore, the carboxyvinyl polymer contains carboxylic residue in the ratio of 56.0 to 68.0% regardless of a kind of polymer including Carbomer 934, Carbomer 934P, Carbomer 940, Carbomer 941 or Carbomer 947P. The content of carboxyvinyl polymer is within the range of 0.001 to 50 wt % on the basis of the total weight of the composition, preferably 0.005 to 25 wt %, more preferably 0.01 to 10 wt %.

The composition according to the present invention may further contain pharmaceutically acceptable additives, for example stabilizer, excipient, isotropic agent, moisturizing agent, pH controlling agent and so on.

The present inventors have conducted the stability test comparing the EGF preparation containing the carboxyvinyl polymer according to the present invention with EGF preparations containing another polymers as a base for six months at 4° C. and 25° C. In this case EGF dissolved in 10 mM phosphate buffer is used as a control and the content of EGF is analyzed with ELISA method. As a result, EGF preparation containing the carboxyvinyl polymer as a base according to the present invention shows a significant stabilization in the various concentration as compared with EGF preparations containing another base as well as EGF dissolved in phosphate buffer. From this result, it is identified that EGF in EGF preparation according to the present invention is stabilized by the addition of the carboxyvinyl polymer regardless of contents thereof and then the polymer may be used as a base controlling its viscosity optionally and be added as a stabilizer depending on the purpose for use.

The composition containing EGF according to the present invention is useful in eye formulations, topical formulations for a skin such as cream, ointment, gel, patch and so on, and the composition may be used by coating or spreading on the cotton plane surface gauze, and the composition can be stored in a lyophilized form and then dissolved in a suitable solvent when it is used if necessary. Furthermore the topical formulation for the skin may be useful in cosmetic formulation.

The present invention is more specifically explained by the following examples. However, it should be understood that the present invention is not limited to these examples in any manner.

### EXAMPLE 1

An eyedrop formulation containing Carbomer(0.1%)

| EGF | 0.5 | mg |
|---|---|---|
| Carbomer 934P | 0.1 | g |
| Mannitol | 5 | g |
| Methyl paraoxybenzoate | 0.04 | g |
| Propyl paraoxybenzoate | 0.01 | g |
| Sodium hydroxide | q.s | |
| Distilled water for injection | q.s | |
| Total | 100 | g |

The formulation was prepared by using the above-mentioned components in given amounts according to a conventional method. Specifically, mannitol, methyl paraoxybenzoate and propyl paraoxybenzoate were dissolved in appropriate amounts of distilled water for injection. Carbomer 934P(BFGoodrich, U.S.A.) was added to the solution and dispersed therein with stirring. Then, the solution was sterilized after controlling pH with sodium hydroxide, and mixed with filtered and sterilized solution of EGF(Daewoong Pharm., Korea) in distilled water for injection to obtain 100 g of formulation.

### EXAMPLE 2

10 mM of phosphate buffer containing EGF

| EGF | 0.5 | mg |
|---|---|---|
| Sodium hydrogen phosphate | 0.14 | g |
| Sodium chloride | 0.88 | g |
| 20% phosphoric acid | q.s | |
| Total | 100 | g |

The formulation was prepared by using the above-mentioned components in given amounts according to a conventional method. Specifically, sodium hydrogen phosphate and sodium chloride were dissolved in appropriate amounts of distilled water for injection, the solution was sterilized after controlling pH with 20% phosphoric acid, and mixed with filtered and sterilized solution of EGF in distilled water for injection to obtain 100 g of formulation.

### EXAMPLE 3

An eyedrop formulation containing sodium
carboxylmethylcellulose (0.5%)

| EGF | 0.5 | mg |
|---|---|---|
| Sodium carboxylmethylcellulose(Sod. CMC) | 0.5 | g |
| Sorbitol | 5.47 | g |
| Methyl paraoxybenzoate | 0.05 | g |
| Sodium hydroxide | q.s | |
| Distilled water for injection | q.s | |
| Total | 100 | g |

The formulation was prepared by using the above-mentioned components in given amounts according to a conventional method. Specifically, sorbitol and methyl paraoxybenzoate were dissolved in appropriate amounts of distilled water for injection, sodium carboxylmethylcellulose was added to the solution and dispersed therein with stirring. Then, the solution was sterilized after controlling pH with sodium hydroxide, and mixed with filtered and sterilized solution of EGF in distilled water for injection to obtain 100 g of formulation.

### EXAMPLE 4

A topical gel formulation containing Carbomer(1%)

| EGF | 5 | mg |
|---|---|---|
| Carbomer 934P | 1 | g |
| Methyl paraoxybenzoate | 0.2 | g |
| Propylene glycol | 20 | g |
| Sodium hydroxide | q.s | |
| Distilled water for injection | q.s | |
| Total | 100 | g |

The formulation was prepared by using the above-mentioned components in given amounts according to a conventional method. Specifically. methyl paraoxybenzoate was dissolved in appropriate amounts of distilled water for injection, Carbomer 934P was added to the solution and dispersed therein with stirring. Then, the pH of the solution was controlled with sodium hydroxide, the solution was blended with propylene glycol and sterilized by heating. Then, filtered and sterilized solution of EGF in distilled water for injection was added thereto to obtain 100 g of formulation.

### EXAMPLE 5

A topical formulation containing Poloxamer(15%)

| EGF | 5 | mg |
|---|---|---|
| Poloxamer 407 | 15 | g |
| Methyl paraoxybenzoate | 0.2 | g |
| Sodium hydrogen phosphate | 272.18 | mg |
| Sodium chloride | 666.22 | mg |
| Phosphoric acid | q.s | |
| Propylene glycol | 20 | g |
| Distilled water for injection | q.s | |
| Total | 100 | g |

The formulation was prepared by using the above-mentioned components in given amounts according to a conventional method. Specifically, phosphate buffer was prepared by using sodium hydrogen phosphate, sodium chloride and phosphoric acid in given amounts. Methyl paraoxybenzoate as the preservative was dissolved to the phosphate buffer. Poloxamer 407(BASF, Germany) was added to the solution and dispersed therein with string. Then the solution was blended with propylene glycol, dispersed therein with stirring. Then, the pH of the solution was controlled with sodium hydroxide, the solution was blended with propylene glycol and sterilized by heating. Then, filtered and sterilized solution of EGF in distilled water for injection was added thereto to obtain 100 g of formulation.

### Example 5

A topical formulation containing Poloxamer(15%)

| EGF | 5 | mg |
|---|---|---|
| Poloxamer 407 | 15 | g |
| Methyl paraoxybenzoate | 0.2 | g |
| Sodium hydrogen phosphate | 272.18 | mg |
| Sodium chloride | 666.22 | mg |
| Phosphoric acid | q.s | |
| Propylene glycol | 20 | g |
| Distilled water for injection | q.s | |
| Total | 100 | g |

The formulation was prepared by using the above-mentioned components in given amounts according to a conventional method. Specifically, phosphate buffer was prepared by using sodium hydrogen phosphate, sodium chloride and phosphoric acid in given amounts. Methyl paraoxybenzoate as the preservative was dissolved to the phosphate buffer. Poloxamer 407(BASF, Germany) was added to the solution and dispersed therein with stirring. Then the solution was blended with propylene glycol, and then EGF as the active ingredient was added thereto to obtain 100 g of the formulation.

### EXAMPLE 6

A cream formulation containing Carbomer(0.1%)

| EGF | 0.05 | mg |
|---|---|---|
| Glycerin | 4.5 | g |
| Methyl paraoxybenzoate | 0.15 | g |
| Propyl paraoxybenzoate | 0.05 | g |
| Carbomer 940 | 0.1 | g |
| Steary alcohol | 1.75 | g |
| Cetyl alcohol | 4.00 | g |
| Span #60 | 0.50 | g |
| Polyoxyl #40 stearate | 2.00 | g |
| Triethanolamine | q.s | |
| Distilled water for injection | q.s | |
| Total | 100 | g |

The formulation were prepared by using the above-mentioned components in oven amounts according to a conventional method. Specifically, glycerin and methyl paraoxybenzoate were dissolved in appropriate amounts of distilled water for injection, Carbomer 940(BF Goodrich, U.S.A.) was added to the solution and dispersed therein with stirring. Then, propyl paraoxybenzoate and the others were added to the solution and emulsified with melting. Then, the solution was sterilized after controlling pH with triethanolamine, and mixed with filtered and sterilized solution of EGF(Daewoong Pharm., Korea) in distilled water for injection to obtain 100 g of formulation.

### EXAMPLE 7

An ointment formulation containing Carbomer(0.1%)

| EGF | 0.5 | mg |
|---|---|---|
| Methyl paraoxybenzoate | 0.10 | g |
| Propyl paraoxybenzoate | 0.05 | g |
| Carbomer 940 | 0.1 | g |
| Beeswax | 5 | g |
| Mineral oil | 45 | g |
| Borax | 0.2 | g |
| Microcrystalline wax | 7.00 | g |
| Paraffin wax | 10 | g |
| Distilled water for injection | q.s | |
| Total | 100 | g |

The formulation was prepared by using the above-mentioned components in given amounts according to a conventional method. Specifically, methyl paraoxybenzoate, propyl paraoxybenzoate and Carbomer 940(BF Goodrich U.S.A.) were dissolved and dispersed in appropriate amounts of distilled water for injection. The rest waxes were added to the solution and emulsified at an elevated temperature. Then, the solution was sterilized by emulsifying. and mixed with filtered and sterilized solution of EGF(Daewoong Pharm., Korea) in distilled water for injection to obtain 100 g of formulation.

### EXAMPLE 8

A patch formulation containing Carbomer(1%)

| EGF | 1.0 | mg |
|---|---|---|
| Polyvinylalcohol | 20 | g |
| Polyvinylpyrrolidone | 15 | g |
| Carbomer 940 | 1 | g |
| Polyethyleneglycol 4000 | 5 | g |
| Glycerol | 3 | g |
| Distilled water for injection | q.s | |
| Total | 100 | g |

The formulation was prepared by using the above-mentioned components in given amounts according to a conventional method. Specifically, Carbomer 940(BF Goodrich, U.S.A.), polyvinylalcohol, polyvinylpyrrolidine, PEG 400, Glycerol were dissolved and dispersed in appropriate amounts of distilled water for injection. The solution was sterilized at an elevated temperature, and mixed with filtered and sterilized solution of EGF (Daewoong Pharm., Korea) in distilled water for injection to obtain 100 g of formulation. Then, the solution was pour into the mold to form the patch.

Experiment 1

Stability Test of Eyedrop Formulation

The stability of eyedrop formulation containing Carbomer prepared in Example 1 was tested as compared with the carboxyl methyl cellulose-containing formulation prepared in Example 2 which was known to stabilize EGF. The test was conducted to estimate EGF contents of each formulation with the lapse of time(2, 4, 8 and 18 weeks) under storage at 4° C. and 25° C. The sample of Example 2 dissolved in 10 mM phosphate buffer was used to standard sample and the content of EGF was estimated by ELISA Method of Quantikne EGF ELISA kit(R&D, U.S.A.).

Table 1 shows the result regarding the stability of EGF-containing eyedrop formulation as compared with standard sample at 4° C. and Table 2 shows the result regarding the stability of EGF-containing eyedrop formulation as compared with standard sample at 25° C .

As can be seen from the below Table 1, EGF content in phosphate buffer was decreased by about 10% in 8 weeks at 4° C. while EGF contents in Carbomer and carboxyl methyl cellulose were not changed until 8 weeks. However, in storage of 18 weeks at 4° C. condition, EGF contents in phosphate buffer and Carbomer formulation were not changed but EGF content in the carboxyl methyl cellulose was decreased to 87.3% in 18 weeks.

TABLE 1

| | Initial | conc. (%) at 4° C. | | | |
|---|---|---|---|---|---|
| Sample | conc. (%) | 2 weeks | 4 weeks | 8 weeks | 18 weeks |
| Example 1 | 100 ± 2.5 | 99.2 ± 3.2 | 102.0 ± 4.3 | 103.7 ± 1.2 | 101.6 ± 3.5 |
| 0.1% Carbomer | | | | | |
| Example 2 | 100 ± 1.9 | 98.4 ± 5.4 | 96.8 ± 14.0 | 91.6 ± 10.3 | 92.5 ± 5.9 |
| 10 mM phosphate | | | | | |
| buffer | | | | | |
| Example 3 | 100 ± 2.1 | 104.9 ± 3.4 | 99.7 ± 6.0 | 102.7 ± 2.3 | 87.3 ± 3.1 |
| 0.5% sodium | | | | | |
| carboxyl methyl | | | | | |
| cellulose | | | | | |

As can be seen from the below Table 2, when the same formulations were stored at 25° C., the content of EGF in phosphate buffer sample was decreased by about 20% in 2 weeks and the content of EGF was continuously decreased after 4 weeks in the case of carboxyl methyl cellulose. However, the content of EGF in the formulation of Example 1 was little changed until 8 weeks. Also, when the formulation of Example 1 was stored for 18 weeks at the room temperature. the content of EGF was decreased by about 13% only. Therefore, it was identified that EGF stability was significantly increased even under storage at the room temperature in case of formulation containing Carbomer.

TABLE 2

| | Initial | conc. (%) at 25° C. | | | |
|---|---|---|---|---|---|
| Sample | conc. (%) | 2 weeks | 4 weeks | 8 weeks | 18 weeks |
| Example 1 | 100 ± 2.5 | 98.2 ± 2.5 | 101.8 ± 2.4 | 101.8 ± 2.4 | 87.6 ± 5.2 |
| 0.1% Carbomer | | | | | |
| Example 2 | 100 ± 1.9 | 81.6 ± 3.6 | 88.4 ± 6.9 | 81.3 ± 1.7 | 72.5 ± 3.3 |
| 10 mM phosphate | | | | | |
| buffer | | | | | |
| Example 3 | 100 ± 2.1 | 93.5 ± 6.5 | 88.4 ± 0.2 | 78.5 ± 2.7 | 48.7 ± 9.3 |
| 0.5% sodium | | | | | |
| carboxyl methyl | | | | | |
| cellulose | | | | | |

Experiment 2
Stability Test of Topical Gel Formulation

The stability of topical gel formulation prepared in Example 4 was tested as compared with the topical formulation containing Poloxamer being widely used as a base for topical formulation which is a neutral polymer and is known to contribute to stabilization of protein resulting from lowering dielectric constant in an aqueous solution. The test was conducted to estimate EGF content of each formulation in storage in 18 weeks at 4° C. and 25° C. The sample dissolved in 10 mM phosphate buffer was used to standard sample and the content of EGF was estimated by ELISA Method of Quantikine EGF ELISA kit(R&D, U.S.A.).

Table 3 and 4 show the stability of each topical gel formulation at 4° C. 15 and 25° C. respectively. As can be seen from the below Table 3, EGF content of the formulations containing Carbomer or Poloxamer was not changed until 8 weeks in cold storage. However, in storage for 18 weeks, EGF content of Poloxamer-containing formulation was decreased by about 10%. As can be seen from the below Table 4, EGF content of 1% Carbomer-containing formulation was little changed until 18 weeks while EGF content of Poloxamer-containing formulation or phosphate buffer formulation was decreased by about 20% in 8 weeks and then continuously decreased until 18 weeks. The degree of decrease was further large in the case of Poloxamer-containing formulation. As seen from the eyedrop formulation, when a polymer was used as a base, the content of EGF was further decreased rather than phosphate buffer as time passed because the polymer might further promote the degradation of EGF in long-term storage. In conclusion, it was identified that the stability of EGF in formulation could be improved by using Carbomer as a base necessarily.

TABLE 3

| | Initial | conc. (%) at 4° C. | | | |
|---|---|---|---|---|---|
| Sample | conc. (%) | 2 weeks | 4 weeks | 8 weeks | 18 weeks |
| Example 2 | 100 ± 1.9 | 98.4 ± 5.4 | 96.8 ± 14.0 | 91.6 ± 10.3 | 92.5 ± 5.9 |
| 10 mM phosphate | | | | | |
| buffer | | | | | |
| Example 4 | 100 ± 1.8 | 104.5 ± 14.2 | 102.3 ± 2.6 | 101.2 ± 0.8 | 100.3 ± 2.3 |
| 1% Carbomer | | | | | |
| Example 4 | 100 ± 2.8 | 103.5 ± 9.3 | 95.7 ± 0.8 | 94.2 ± 4.2 | 90.5 ± 4.5 |
| 15% Poloxamer | | | | | |

TABLE 4

| | Initial | conc. (%) at 25° C. | | | |
|---|---|---|---|---|---|
| Sample | conc. (%) | 2 weeks | 4 weeks | 8 weeks | 18 weeks |
| Example 2 | 100 ± 1.9 | 81.6 ± 3.6 | 88.4 ± 6.9 | 81.3 ± 1.7 | 72.5 ± 3.3 |
| 10 mM phosphate | | | | | |
| buffer | | | | | |
| Example 4 | 100 ± 1.8 | 107.3 ± 2.0 | 92.5 ± 0.5 | 101.8 ± 2.4 | 99.5 ± 4.5 |
| 1% Carbomer | | | | | |
| Example 5 | 100 ± 2.8 | 90.3 ± 41.4 | 79.5 ± 5.0 | 78.5 ± 2.7 | 66.4 ± 2.6 |
| 15% Poloxamer | | | | | |

Experiment 3
Stability Test of Cream, Ointment and Patch Formulations

To estimate the stability of Carbomer-containing formulations prepared in Examples 6, 7, and 8, the test was conducted to estimate EGF content of each formulation with the lapse of time(2, 4, 8 and 18 weeks) under storage at 4° C. and 25° C. The sample of Example 2 dissolved in 10 mM phosphate buffer was used to standard sample and the content of EGF was estimated by ELISA Method of Quantikine EGF ELISA kit(R&D, U.S.A.).

Table 5 and 6 shows the stability of each cream, ointment and patch formulation at 4° C. and 25° C. respectively. As can be seen from the below Table 5, EGF content was not changed in cold storage. As can be seen from the below Table 6, EGF content was little changed at a room temperature. is Therefore. it was identified that the stability of EGF in the formulation could be improved by using Carbomer as a base regardless of the type of formulation.

TABLE 5

| | Initial | conc. (%) at 4° C. | | | |
|---|---|---|---|---|---|
| Sample | conc. (%) | 2 weeks | 4 weeks | 8 weeks | 18 weeks |
| Example 6 | 100 ± 2.9 | 99.6 ± 5.2 | 102.5 ± 7.2 | 101.4 ± 1.9 | 97.9 ± 6.4 |
| 0.1% Carbomer | | | | | |
| cream | | | | | |
| Example 7 | 100 ± 2.3 | 97.0 ± 9.5 | 100.1 ± 5.7 | 98.9 ± 2.1 | 98.5 ± 3.3 |
| 0.1% Carbomer | | | | | |
| ointment | | | | | |
| Example 8 | 100 ± 3.5 | 98.5 ± 6.5 | 97.4 ± 8.6 | 98.4 ± 2.7 | 97.5 ± 5.8 |
| 1% Carbomer patch | | | | | |

TABLE 6

| | Initial | conc. (%) at 25° C. | | | |
|---|---|---|---|---|---|
| Sample | conc. (%) | 2 weeks | 4 weeks | 8 weeks | 18 weeks |
| Example 6 | 100 ± 2.9 | 100.1 ± 6.2 | 100.5 ± 3.3 | 96.7 ± 2.5 | 95.2 ± 4.5 |
| 0.1% Carbomer | | | | | |
| cream | | | | | |
| Example 7 | 100 ± 2.3 | 99.5 ± 6.3 | 102.1 ± 5.1 | 94.8 ± 1.8 | 96.2 ± 8.9 |
| 0.1% Carbomer | | | | | |
| ointment | | | | | |
| Example 8 | 100 ± 3.5 | 100.2 ± 12.3 | 96.5 ± 9.4 | 97.2 ± 8.8 | 95.7 ± 8.4 |
| 1% Carbomer patch | | | | | |

As shown in the results obtained from the above experiments, the present invention provides a stable EGF composition, which comprises carboxyvinyl polymers as a base and biologically active EGF of which the stability is biologically and physicochemically ensured.

1. A method for stabilizing a composition having a biologically active EGF as an active ingredient, comprising adding a carboxyvinyl polymer as a stabilizer of the EGF, wherein the content of the EGF is within the range of 0.1 to 100 μg/g on the basis of the total weight of the composition, and the content of the carboxyvinyl polymer is within the range of 0.001 to 50 (w/w) % on the basis of the total weight of the composition.

2. The method according to claim 1, wherein the biologically active EGF is isolated from natural sources or produced by using recombinant DNA techniques.

3. The method according to claim 1, wherein the pH of the composition in an aqueous solution is within the range of 4 to 8.

4. The method according to claim 1, wherein the carboxyvinyl polymer is selected from the group consisting of Carbomer 934, Carbomer 934P, Carbomer 940, Carbomer 941 or Carbomer 947P.

5. The method according to claim 1, wherein the content of the carboxyvinyl polymer is within the range of 0.005 to 25(w/w) % on the basis of the total weight of the composition

6. The method according to claim 1, wherein the content of the carboxyvinyl polymer is within the range of 0.01 to 10 (w/w) % on the basis of the total weight of the composition.

7. The method according to claim 1, wherein the composition is an eye formulation.

8. The method according to claim 1, wherein the composition is a topical formulation.

9. The method according to claim 8, wherein the composition is a cream formulation.

10. The method according to claim 8, wherein the composition is an ointment formulation.

11. The method according to claim 8, wherein the composition is a gel formulation.

12. The method according to claim 8, wherein the composition is a patch formulation.

13. The method according to claim 8, wherein the composition is spread on a cotton plane surface or gauze.
